(19) United States
(12) Patent Application Publication
(10) Pub. No.: US 2024/0122482 A1
(43) Pub. Date: Apr. 18, 2024

(54) Method and System for Detecting the State of Health of a Person

(71) Applicant: Seiwo Technik GmbH, Drehbach, OT Scharfenstein (DE)

(72) Inventors: Jan Wabst, Chemnitz (DE); Thomas Krause, Neumark (DE)

(21) Appl. No.: 18/279,077

(22) PCT Filed: Feb. 9, 2022
(86) PCT No.: PCT/DE2022/100110

§ 371 Date: Aug. 28, 2023

(30) Foreign Application Priority Data
Feb. 26, 2021 (DE) 202021100976.4

(51) Int. Cl. A61B 5/01; A61B 5/00; A61B 5/0205

(52) U.S. Cl. CPC A61B 5/01; A61B 5/0006; A61B 5/002; A61B 5/02055; A61B 5/681; A61B 5/7267; A61B 2562/0271; A61B 2562/043

(57) ABSTRACT

The invention relates to a method for detecting the state of health of a person, said method having the steps of detecting the body temperature of the person, transmitting the detected body temperature values to an evaluation unit, and evaluating the body temperature values by means of the evaluation unit, the body temperature being detected at a frequency of at least one measurement every 10 minutes. The invention also relates to a system for detecting the state of health of a person, said system comprising at least one sensor for detecting the body temperature and at least one evaluation unit to which the data detected by the sensor are transmitted, the sensor being suitable for detecting the body temperature at least once every 10 minutes and transmitting same to the evaluation unit.

### CROSS REFERENCE TO RELATED APPLICATIONS

This application is the U.S. national stage of International Application No. PCT/DE2022/100110, filed on 2022 Feb. 9. The international application claims the priority of DE 202021100976.4 filed on 2021 Feb. 26; all applications are incorporated by reference herein in their entirety.

### BACKGROUND

The present invention relates to a method for detecting the state of health of a person. In addition, the present invention relates to a system for detecting the state of health of a person.

Many diseases are already transmissible during their incubation period, i.e. the time that elapses between infection with a pathogen and the appearance of the first symptoms. Since during this time the infection of a person is usually not yet recognizable, no preventive hygiene measures are usually taken to prevent infection of other people, such as keeping sufficient distance from other people or refraining from shaking hands. In particular, diseases that can be transmitted by droplet infection, direct contact or smear infection can thus spread quickly among a group of people without being noticed.

In conventional flu, an infected person is already contagious during the incubation period, which lasts up to three days. In the case of a cold, infection can also occur during the incubation period, which lasts two to eight days. In the case of the coronavirus SARS-CoV-2, which can cause the disease Covid-19, the incubation period is two to 14 days, on average five to six days.

Early detection of infections is therefore important to prevent the spread of pathogens.

WO 2016 037 091 A1 describes a computer-implemented method and associated system for monitoring the well-being of a person by providing a pair of eyeglasses that includes at least one sensor for monitoring the movement of the user. In various embodiments, the system receives data generated by the at least one sensor, uses the data to determine the user's movements based on the received data, and compares the user's movements to predetermined movement patterns of the user. If the system detects one or more inconsistencies between the user's current movements compared to the user's previously determined movement patterns, the system may notify the user or a third party of the detected inconsistency or inconsistencies. The system may similarly monitor the user's compliance with a medical regimen and notify the user or a third party of the user's compliance with the regimen.

U.S. Pat. No. 2,014,316 220 A1 describes a personal health monitoring device that includes a memory for collecting and storing attributes of an individual and a processor for quantizing each attribute in such a way as to indicate a normal range for that attribute and for measuring deviations from that normal range. The processor also calculates the well-being of the individual based on the measured deviations. The results are displayed and indicate the well-being of the individual.

DE 10 2013 222 123 A1 describes a mobile monitoring device for a body function of a living being, comprising a detection area, an attachment element, a sensor and an evaluation unit. The detection area is designed to detect at least one analyte. The attachment element is configured to bring the detection area into contact with a body surface of the living being to be monitored. The sensor is coupled to the detection area to obtain the at least one analyte, and is configured to output an electrical signal in response to the at least one analyte obtained. The evaluation unit is configured to determine information about the at least one analyte based on the electrical signal from the sensor.

The publication DE 10 2014 006 000 A1 describes a device and method for in vivo acquisition of patient data and transmission to a data processing device. A device for temperature measurement in patients is to be provided, which enables a simple and cost-effective clinical temperature measurement in patients. This is accomplished by means of a temperature measurement adapter, having a metallic contact element and a medical adhesive tape for adhering to the patient's skin. A contact protrusion is provided for thermally conductive coupling of a temperature measuring device. The temperature measurement adapter is adhered to the patient's skin. It measures the skin temperature by means of only one temperature sensor via the adapter and transmits the data by radio. The detecting of the body temperature is not realized.

In the article “Wireless Vital Signs Monitoring. Cadi Scientific Pte Ltd, November 2016. URL: https://cadi.com.sg/2016/11/12/wireless-vital-signs-monitoring/archived in http://www.archive.org on Aug. 9, 2020 [accessed Oct. 18, 2021],” a solution for measuring vital signs, including temperature measurement, of patients in hospitals is also described. A mobile vital signs monitoring device is disclosed that can be used to monitor the vital signs of patients in inpatient settings. This solution is not suitable for the outpatient setting and cannot be used in everyday life. The parts of the measurement system interfere with the wearer in everyday life. The system does not record body temperature. Only the skin temperature is measured by means of a temperature sensor. The system does not perform any evaluation. The detected measured values are only displayed and stored.

WO 2016/164375 A1 describes a system for assessing the emotional state of an infant. The starting point is audiovisual monitoring systems for babies and toddlers. Only temperature and movement are detected as vital signs, but no body temperature is measured. Furthermore, no evaluation of the state of health takes place. Another disadvantage is that this system can only be used stationary and is only suitable for toddlers and babies.

The publication CN 2 11 147 894 U describes a mobile system for detecting temperature and its transmission and storage on a central system (server). The system is to be used as an alternative to manual stationary temperature measurement. It also records only skin temperature by means of a single sensor and not body temperature. The sensor technology described is very inaccurate with a tolerance of over 0.7 K.

Finally, DE 11 2018 005 619 T5 provides a software, a health condition determination device, and a health condition determination method that reflect vital parameters or daily conditions while taking into account individual differences of subjects to enable subjects to detect intraindividual variation with high accuracy and contribute to health care of subjects or provision of medical care for characteristics of each individual.

However, with all known solutions, it is not possible to detect an infection in a person already during the incubation period, during which there are still no symptoms that are clearly visible from the outside.

### SUMMARY

The invention is thus based on the object of providing a method and a system with which potential infections of a person can be detected and reported at an early stage and which neither disturb nor influence the person in their daily routines.

This object is solved on the one hand by a method for detecting the state of health of a person, wherein the body temperature of the person is detected, wherein two or more temperature sensors are used in parallel and at least one temperature sensor is a skin sensor, the detected body temperature values are transmitted to an evaluation unit and an evaluation (6) of the body temperature values is carried out by the evaluation unit (5), wherein the detection of the body temperature takes place with a frequency of at least one measurement every 10 minutes and wherein for the evaluation (6) of the body temperature values a comparison of the detected body temperature values with previously determined standard values of the body temperature of the person is carried out.

### DETAILED DESCRIPTION

Surprisingly, it has been recognized that the body temperature of an infected person can rise unnoticed even during the symptom-free incubation period. Regular detection of body temperature thus offers the possibility of detecting infections before the outbreak of the actual disease and avoiding infection of other people.

Advantageously, the body temperature is detected at very short intervals, preferably with a frequency of at least one measurement every 10 minutes. In particular, such measurements are integrated into the person's daily routine and are carried out permanently without impairing or disturbing the person. For this purpose, it is particularly suitable to detect the body temperature by means of a device that can be worn inconspicuously on the body, such as a wristband, a fitness tracker, a watch or also via sensors that can be integrated into clothing such as underwear or the like.

In a preferred embodiment of the method according to the invention, the body temperature is detected at a frequency of at least one measurement per minute.

Such close-meshed measurement intervals make it possible to quickly register and report deviations from the person's usual body temperature or from predefined values so that the person can respond with measures.

Preferably, body temperature is measured with an accuracy of +/−0.1° C.

It may be provided that two or more temperature sensors are used in parallel to carry out the method according to the invention. This increases the accuracy of the method and makes it possible to determine average values for each measuring point, which can then be analyzed further.

In particular, at least one skin sensor is used to carry out the method according to the invention. Conclusions about the core body temperature can be drawn from the detected surface temperature of the person.

Advantageously, the evaluation of the body temperature values takes place on a server.

In particular, this enables the body temperature values to be detected centrally. Preferably, the complete analysis is performed on the server. Access to the data and the results of the analysis is thus advantageously possible from various end devices. The data and/or the results of the analysis can be displayed in a web browser or via an app, for example.

It may be provided that the evaluation of the body temperature values is carried out by means of neural networks.

It may be further provided that for the evaluation of the body temperature values an evaluation algorithm is used, which is an analytical algorithm or a self-learning algorithm.

Advantageously, a combination of the results of several algorithms can be made into an infection prediction.

It is conceivable that a temperature curve is created to evaluate the body temperature values.

In particular, for the evaluation of the body temperature values, a comparison is made between the detected body temperature values and previously determined standard values of the person's body temperature. In this way, a “normal” state of a person's body temperature can be determined and defined, from which an “abnormal” state can be clearly differentiated.

It may be provided that, in addition to the body temperature, the pulse of the person and/or the oxygen content of the blood of the person and/or a movement pattern of the person and/or the blood pressure of the person and/or the breathing rate of the person and/or the skin resistance of the person and/or the time and/or duration of sleep phases and/or the sleep rhythm of the person are detected.

It is conceivable that for the duration of sleep phases or during phases of increased physical intensity, for example during detected or manually specified sport units, the tightness of the measurement intervals is reduced, i.e. the interval between measurements is increased. For example, it would be conceivable to interrupt the measurement of body temperature for half an hour or for a manually determinable period for the duration of a sports unit.

Furthermore, there is also a connection between the pulse and the body temperature of a person. A slightly elevated resting pulse, for example, may indicate that the body has to make an extra effort to keep the body temperature in the normal range and may therefore also be associated with an infection.

The object is further solved by a system for detecting the state of health of a person, which is designed as a system to be worn on the wrist of a person, comprising two or more temperature sensors for parallel detection of the body temperature, wherein at least one temperature sensor is a skin sensor, at least one evaluation unit to which the data detected by the sensors are transmitted for evaluation of the body temperature values by comparison of the detected body temperature values with previously determined standard values of the body temperature of the person, wherein the sensors are suitable for detecting the body temperature at least once every 10 minutes and transmitting it to the evaluation unit.

Parallel measurement of two or more sensors increases the accuracy and provides the possibility to form average values of the detected temperature.

In a preferred embodiment of the system according to the invention, the sensor is suitable for detecting the body temperature at least once per minute and transmitting it to the evaluation unit.

Due to the tightness of the measurement intervals with measurements every 10 minute, preferably every minute, deviations of the body temperature values from a “normal state” of the person's body temperature or from predefined standard values are perceived promptly.

Advantageously, the system is designed to be worn on the wrist of a person. For example, it may be designed as a watch, fitness watch or wristband.

Alternatively, the system according to the invention can be integrated in a person's clothing, for example in underwear, sports underwear or other functional clothing.

Alternatively, the system according to the invention can be worn on other parts of the body than the wrist, for example the ankle or hip.

Advantageously, an unobtrusive design of the system according to the invention (light, space-saving, etc.) makes it possible to integrate the measurements into the person's everyday life and to carry them out permanently without interfering with or disturbing the person.

The evaluation unit of the system according to the invention is preferably connected to a server on which the analysis of the detected body temperature values takes place. The evaluation unit can be, for example, a smartphone, a computer, a gateway or the like. Access to the data and the results of the analysis is advantageously possible from various terminal devices. A display of the data and/or the results of the analysis can take place, for example, in a web browser or via an app.

Preferably, the evaluation unit is suitable for performing the analysis of the body temperature values by means of neural networks. The evaluation can be performed using an analytical algorithm or a self-learning algorithm.

The evaluation unit preferably generates a temperature curve from the determined body temperature values and permanently compares these with previously determined standard values of the person's body temperature or with predefined values. In this way, deviations from standard values or from predefined values are determined promptly as they occur and can be reported by the evaluation unit.

It may be provided that the system according to the invention further comprises an acceleration sensor for detecting a movement pattern and/or the sleep phases and/or the sleep rhythm of the person.

It is conceivable that for the duration of sleep phases or during phases of increased physical intensity, for example during detected or manually specified sport units, the tightness of the measurement intervals is reduced, i.e. the interval between measurements is increased. For example, it would be conceivable to interrupt the measurement of body temperature for half an hour or for a manually determinable period for the duration of a sports unit.

It may further be provided that the system according to the invention further comprises a heart rate monitor and/or a blood pressure monitor and/or an oxygen sensor and/or a respiration rate monitor and/or a skin resistance monitor and/or a means for detecting the time and/or for detecting or displaying the date and/or an NFC chip.

In one embodiment, the system according to the invention may further comprise a Bluetooth transmitter and/or a Bluetooth receiver.

Preferably, the system according to the invention also comprises means for localizing the sensor, for example by detecting the affiliation to radio cells or an integrated GPS receiver or UWB GPS receiver.

It may be provided that the system according to the invention comprises an SOS button for easy sending of a call for help.

It may further be provided that a distance measurement is carried out between different persons wearing a wearable means for detecting body temperature (for example a smartwatch) belonging to the system according to the invention. In the event of an infection, this makes it possible to interrogate chains of contact and quarantine measures can thus be significantly smaller.

Advantageously, the method and system according to the invention make it possible to permanently monitor the body temperature of a person and to promptly detect and report deviations from a normal state. Since an increase in body temperature may be associated with an as yet undetected infection of the person, the person can take preventive measures, such as minimizing contact with fellow humans. Indications of a possible infection are possible by the method and system according to the invention well before symptoms appear. Advantageously, a reduction of the unnoticed infection time is made possible and chains of infection are interrupted. In this way, the spread of viruses is advantageously contained.

As particularly advantageous, the method and system according to the invention can be used in old people's homes, nursing homes and in assisted living facilities, but also in schools and universities or in other places where large crowds of people are regularly present. For example, it would be conceivable for caregivers to receive an alert if a person develops circulatory problems, and the alert could include the person's location. Also conceivable are health monitoring at events or prevention of infection of employees in industry and commerce.

When using the system according to the invention in production facilities and industry, it is also conceivable that the device worn on the body can be used to issue messages, for example, about the material requirements of a machine or the need for entries in or actions on a machine. It is also possible to provide feedback and confirmation of stock withdrawals, messages and work steps, automatic logon to workstations or machines with the integrated NFC tag, a message to colleagues or security personnel if an employee develops health problems, or help messages via an SOS button.

Access control for rooms and/or lockers is also conceivable.

For example, the invention can also ensure a normal daily life in the Corona pandemic. It will allow a school run to return to normal, events and gatherings to resume, and mortality rates to decrease. It is conceivable that children, teachers, and staff will wear a fitness tracker or smartwatch with a sensor to track body temperature on a regular basis. Via a Bluetooth gateway or smart device, the data is transmitted to a German server, which analyzes it and detects infection risks. This can lead to predominantly healthy people being present at the schools, who can once again move freely.

Unlike other technologies that require individuals to be checked to stand at a checkpoint and have their temperature and health conditions assessed as they pass, the method and system of the invention enable health monitoring from any location and at any time with real-time data. Health incidents are sent to the platform for further and immediate action.

In the method and system according to the invention, medical grade sensors are used to monitor the wearer's health condition and provide higher accuracy. The measurement accuracy for body temperature is 0.1° C., providing the best accuracy compared to other body temperature measurement devices on the market. This can be the difference between the successful early detection of the virus and the misinterpretation of symptoms.

In addition to health monitoring, the method and system according to the invention provide a variety of activity tracking functions ranging from sleep, walking, steps, running, cycling to gym workouts. All can be displayed, for example, via a mobile app for smartphones. The device with the sensor for detecting the body temperature can receive various mobile notifications of incoming texts, emails, phone calls and more.

### BRIEF DESCRIPTION OF THE DRAWINGS

The invention is explained in more detail below with reference to exemplary embodiments and associated figures, without being limited to the latter, wherein:

FIG. 1 shows a sequence of an embodiment of the method according to the invention for detecting the state of health of a person;

FIG. 2A shows a smartwatch as part of a system according to the invention for detecting the state of health of a person;

FIG. 2B shows a fitness tracker as part of a system according to the invention for detecting the state of health of a person; and

FIG. 3 shows a schematic representation of two possible embodiments of the method according to the invention.

### DETAILED DESCRIPTION OF THE PREFERRED EMBODIMENTS

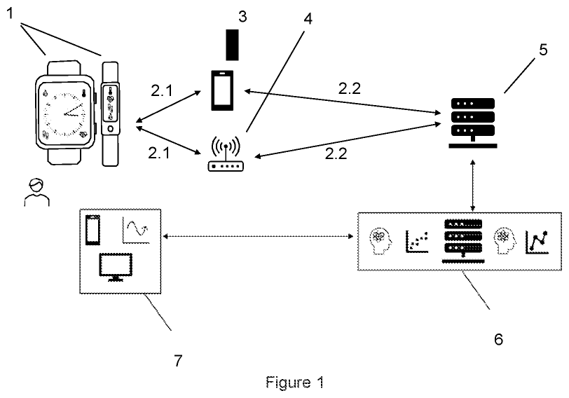

FIG. 1 schematically shows an exemplary sequence of an embodiment of the method according to the invention for detecting the state of health of a person. First, the body temperature values of a person are detected by means of a fitness tracker 1 or a smartwatch 1 with a sensor for detecting the body temperature of a person. In this case, the detection is carried out up to 1440 times per day. The detected values are then transmitted to an external provider 3 or to a home network 4 2.1. The values are further transmitted to an evaluation unit 5, for example a server 2.2. The data can be stored on the server or the evaluation unit 5. Subsequently, the evaluation 6 of the data takes place. The evaluation 6 can be carried out by means of AI (artificial intelligence) or via adaptive algorithms and can also be carried out in relation to Big Data. This is followed by a visualization 7 for the user. Here, both detected basic data and results of the analysis 6 can be visualized. The visualization 7 can take place, for example, on a smartphone, in a web browser or directly on the fitness tracker 1 or the smartwatch 1 with sensor for detecting the body temperature. It can be provided that a message or visualization only occurs when predetermined limit values or limit values defining the normal state are exceeded. For example, a message via a vibration alarm is also conceivable.

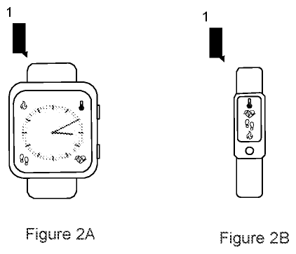

FIGS. 2A and 2B show examples of a smartwatch (FIG. 2A) and a fitness tracker (FIG. 2B), each as part of a system according to the invention for detecting a person's state of health. The smartwatch and the fitness tracker, respectively, may have a screen and a display element, respectively, for displaying various parameters, for example, date, time of day, pulse of the person, body temperature of the person, blood pressure of the person, skin resistance of the person, breathing rate of the person, movement pattern, number of steps, calories burned, sleep pattern, or others. According to the invention, the smartwatch or fitness tracker comprises at least one sensor, not shown in FIGS. 2A and 2B, for detecting the body temperature of a person. The detected body temperature values are transmitted at least once every 10 minutes, preferably at least once per minute, to an evaluation unit where they are stored and/or analyzed. The smartwatch and/or fitness tracker may further comprise an accelerometer for detecting a movement pattern and/or sleep phases and/or sleep rhythm of the person. Likewise, they may comprise a heart rate sensor and/or an oxygen sensor and/or a blood pressure sensor and/or a respiration rate sensor and/or a skin resistance sensor and/or a means for detecting the time of day and/or a Bluetooth transmitter and/or a Bluetooth receiver.

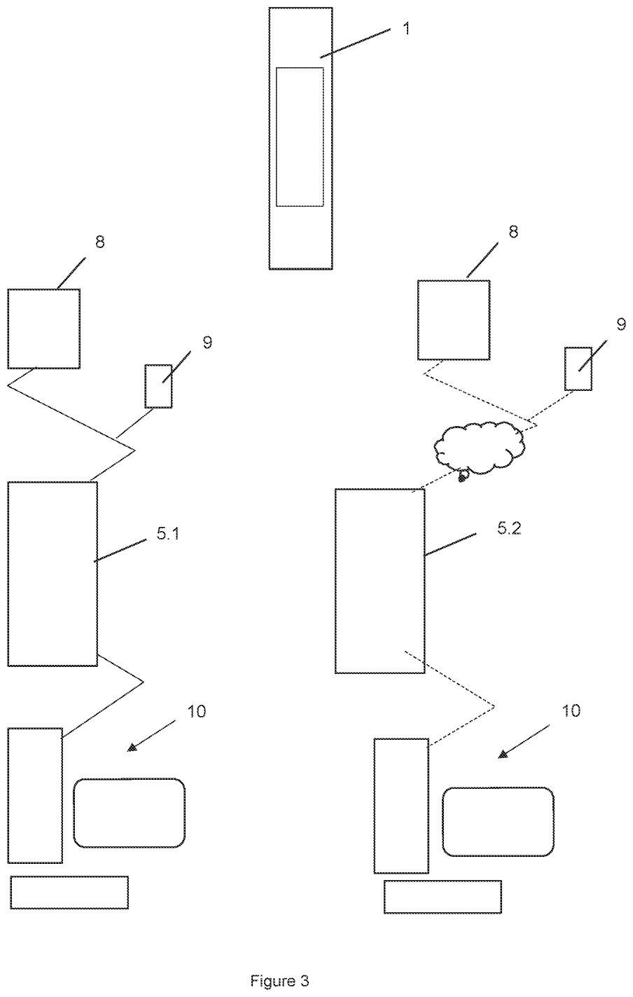

FIG. 3 shows a schematic representation of two possible embodiments of the method according to the invention.

Devices such as a fitness tracker or smartwatch 1 with a sensor for detecting the body temperature are connected to the monitoring platform (the Central Monitoring Workstation 10) via the Smart Bluetooth Gateway 8 or smartphone 9. A user-friendly and intuitive dashboard provides an overview of all connected devices, connection status, real-time alarms and reports, community integrity analysis, and real-time device location. The following alarms are detected: Panic or SOS; abnormal temperature; abnormal heart rate and blood pressure; device not worn; device battery low. The platform not only monitors the user's state of health and activities, but also sends alerts to your system administrator via email and SMS.

All user data is fully secured, either via the cloud or a local solution. FIG. 3 shows the local solution on the left and the cloud-based solution on the right. In the case of the local solution, data is transferred (and stored and analyzed) to a local server 5.1, while in the case of the cloud-based solution, the data transfer (as well as storage and evaluation) is made to a cloud server 5.2.

In the local solution (FIG. 3, left), provisioning takes place on the existing on-site IT infrastructure and all devices are connected to the system provided by the customer. The system is scalable. Devices can be deployed at different locations and connected to the platform via the customer intranet. This option is suitable, for example, for government agencies or companies that are subject to high data protection guidelines and have sufficient infrastructure to operate the system on site.

For the cloud-based solution (FIG. 3 right), all devices are connected to a cloud. The client administrator only needs the Internet to access the cloud platform and monitor and manage the devices.

The cloud-based solution enables connection from anywhere as long as there is an Internet connection. The devices 1 connect to the cloud either via the smart Bluetooth gateway 8 or the smartphone 9. This solution is particularly suitable for individuals and organizations that may not have sufficient infrastructure to host the system on-site.

The solution according to the invention supports different types and levels of integration with third-party systems, depending on the specific user applications. The technology can be used in both stationary and mobile applications. By using the mobile Bluetooth gateway 8, devices 1 can connect and send real-time activity and alarm data, including user location.

In this way, the platform manager can immediately send assistance to the exact location of the healthwatch wearer in the event of a health emergency. By means of NFC technology integrated in some embodiments, the device 1 can be integrated into various organizational applications of hospitals, schools, airports, nursing homes, and many other business environments.

### LIST OF REFERENCE NUMERALS

1 Fitness tracker/smartwatch with sensor for detecting the body temperature
2.1 Transmitting the detected values to an external provider or a home network
2.2 Transmitting the detected values to an evaluation unit
3 External provider
4 Home network
5 Evaluation unit
5.1 Local server
5.2 Cloud server
6 Evaluation
7 Visualization
8 Bluetooth gateway
9 Smart device
10 Central monitoring workstation

1. Method for detecting the state of health of a person, comprising the steps of
detecting the body temperature of the person, wherein two or more temperature sensors are used in parallel and at least one temperature sensor is a skin sensor,
transmission (2.1, 2.2) of the detected body temperature values to an evaluation unit (5), and
evaluation (6) of the body temperature values by the evaluation unit (5),
wherein the detection of the body temperature takes place with a frequency of at least one measurement every 10 minutes and wherein for the evaluation (6) of the body temperature values a comparison of the detected body temperature values with previously determined standard values of the body temperature of the person is carried out.

2. Method according to claim 1, characterized in that the detection of the body temperature is carried out with a frequency of at least one measurement per minute.

3. Method according to claim 1, characterized in that the evaluation (6) of the body temperature values takes place on a server.

4. Method according to claim 1, characterized in that the evaluation (6) of the body temperature values is carried out by means of neural networks.

5. Method according to claim 1, characterized in that for the evaluation (6) of the body temperature values an evaluation algorithm is used, which is an analytical algorithm or a self-learning algorithm.

6. Method according to claim 1, characterized in that, in addition to the body temperature, the pulse of the person and/or the oxygen content of the blood of the person and/or a movement pattern of the person and/or the blood pressure of the person and/or the breathing rate of the person and/or the skin resistance of the person and/or the time and/or duration of sleep phases of the person are detected.

7. System for detecting the state of health of a person, which is designed as a system to be worn on the wrist of a person, comprising
two or more temperature sensors for parallel detection of body temperature, wherein at least one temperature sensor is a skin sensor,
at least one evaluation unit (5) to which the data detected by the sensors are transmitted for evaluation (6) of the body temperature values by comparison of the detected body temperature values with previously determined standard values of the body temperature of the person,
wherein the sensors are suitable for detecting the body temperature at least once every 10 minutes and transmitting it to the evaluation unit (5).

8. System according to claim 7, characterized in that the sensor is suitable for detecting the body temperature at least once per minute and transmitting it to the evaluation unit (5).

9. System according to claim 7, further comprising an accelerometer for detecting a movement pattern and/or sleep phases of the person.

10. System according to claim 7, further comprising a heart rate monitor and/or an oxygen sensor and/or a blood pressure monitor and/or a respiratory rate monitor and/or a skin resistance monitor and/or a means for detecting the time.

11. System according to claim 7, further comprising a Bluetooth transmitter and/or a Bluetooth receiver.
